(19) United States
(12) Patent Application Publication
(10) Pub. No.: US 2026/0279547 A1
(43) Pub. Date: Sep. 17, 2026

(54) METHOD FOR PROVIDING INFORMATION ABOUT IN VITRO FERTILIZED EMBRYO SCREENING, AND APPARATUS USING SAME

(71) Applicant: KAI HEALTH INC., Seoul (KR)

(72) Inventors: Hye Jun LEE, Seoul (KR); Hyung Min KIM, Seoul (KR)

(73) Assignee: KAI HEALTH INC., Seoul (KR)

(21) Appl. No.: 19/469,237

(22) PCT Filed: Feb. 26, 2024
(86) PCT No.: PCT/KR2024/002432

§ 371 Date: Sep. 25, 2025

(30) Foreign Application Priority Data
Apr. 3, 2023 (KR) 10-2023-0043679

(51) Int. Cl. G16H 30/40; G06N 3/0464; G06T 7/13; G06V 10/82; G06V 20/69; G16H 30/20

(52) U.S. Cl. CPC G16H 30/40; G06N 3/0464; G06T 7/13; G06V 10/82; G06V 20/69; G16H 30/20; G06T 2207/20084; G06T 2207/30024

(57) ABSTRACT

Provided are a method and an apparatus for providing information about in vitro fertilized embryo screening using embryo images. The method, executed by a processor, includes the steps of: receiving an original embryo image; extracting, from the original embryo image, both an inner cell mass (ICM) image and a trophectoderm (TE) image; converting the original embryo image, the extracted ICM image, and the extracted TE image into a binary image or gray scale images; and by using a convolutional neural network model pre-trained to screen an in vitro fertilized embryo by using converted original embryo images, converted ICM extracted images, and converted TE extracted images as inputs, determining an in vitro fertilized embryo by using the converted original embryo image, the converted ICM extracted image, and the converted TE extracted image as inputs.

### BACKGROUND

### Technical Field

The present disclosure relates to a method for providing information about in vitro fertilized embryo screening and an apparatus using the same.

### Description of the Related Art

Test-tube baby procedure or in vitro fertilization (IVF) is an effective treatment for infertility. Since the birth of the first baby conceived through IVF in 1978, IVF technology has become widespread, but the success rate still remains low at about 22-30%.

The biggest challenge related to this is how to identify the healthiest embryo among artificially fertilized embryos for implantation into the mother's uterus. Identifying successfully fertilized embryos is crucial for a successful pregnancy.

Until now, IVF procedures have screened embryos based on a few morphological characteristics seen at different stages of embryo development. Although this visual screening method is used in many obstetrics and gynecology hospitals to distinguish healthy embryos, it has the disadvantage of providing insufficient information on the entire development process and being subject to the subjective judgment of the attending physician.

Therefore, there is an urgent need for objective and high-performance embryo evaluation methods, such as image analysis tools that may identify the morphological characteristics of embryos with at least 32 cells (blastocyst), to achieve successful in vitro fertilization.

The background technology of the present disclosure was written to facilitate understanding of the present disclosure. It should not be understood that the matters described in the background technology of the disclosure exist as prior art.

### BRIEF SUMMARY

### Technical Problem

Among the embryonic regions, the inner cell mass (ICM) and trophectoderm (TE) are known to be important factors in identifying embryos suitable for in vitro fertilization.

The ICM and TE are surrounded by two distinct irregularly shaped regions known as the zona pellucida and cavity mass, respectively. The shapes of the ICM and TE are highly irregular and exhibit similar forms. Accordingly, distinguishing between the ICM and TE currently requires the subjective judgment and expertise of medical professionals.

Meanwhile, the inventors of the present disclosure recognized that existing artificial intelligence models for embryo identification in IVF are trained on color or binary images of embryos, and furthermore, even when being trained on extracted inner cell mass images and extracted trophectoderm images, they are trained on color images, so that color information may cause low accuracy in embryo identification for in vitro fertilization.

Furthermore, the inventors of the present disclosure recognized that a convolutional neural network model trained on images with adjusted Red, Blue, and Green values in each RGB channel of an original embryo binary image, ICM extracted binary image, and TE extracted binary image could show excellent performance in embryo screening for in vitro fertilization, and developed a method and an apparatus for providing information about in vitro fertilized embryo screening using this.

Accordingly, an object of the present disclosure is to provide a method and an apparatus for screening in vitro fertilized embryo with a convolutional neural network model trained using the original embryo binary image, the ICM extracted binary image, and the TE extracted binary image, respectively.

Objects of the present disclosure are not limited to the objects above, and other objects not mentioned may be clearly understood by those skilled in the art from the description below.

### Technical Solution

In order to achieve the above-described object, a method for providing information about in vitro fertilized embryo screening according to an embodiment of the present disclosure is provided. A method for providing information about in vitro fertilized embryo screening according to an embodiment of the present disclosure is implemented by a processor and includes: receiving an original embryo image; extracting, from the original embryo image, each of an inner cell mass (ICM) image and a trophectoderm (TE) image; converting each of the original embryo image, extracted ICM image, and extracted TE image into a binary image or grayscale image; and determining an in vitro fertilized embryo by using converted original embryo image, converted ICM extracted image, and converted TE extracted image as inputs, by using a convolutional neural network model pre-trained to screen an in vitro fertilized embryo by using the converted original embryo image, the converted ICM extracted image, and the converted TE extracted image as inputs.

According to a feature of the present disclosure, the method for providing information about in vitro fertilized embryo screening according to an embodiment of the present disclosure further includes reconstructing the converted original embryo image, the converted ICM extracted image, and the converted TE extracted image into a single reconstructed image such that each of the images is distinguishable from one another, and the determining may determine an in vitro fertilized embryo with the reconstructed original embryo image, the reconstructed extracted ICM image, and the reconstructed extracted TE image as inputs.

According to another feature of the present disclosure, the method for providing information about in vitro fertilized embryo screening according to an embodiment of the present disclosure further includes receiving the mother's age value; the convolutional neural network model further includes the mother's age value as an input value of the model, and the determining may determine the in vitro fertilized embryo with the reconstructed original embryo image, the reconstructed extracted ICM image, the reconstructed extracted TE image, and the mother's age value as inputs.

According to another feature of the present disclosure, in the method for providing information about in vitro fertilized embryo screening according to an embodiment of the present disclosure, the original embryo image may be an image captured by one or more microscopes selected from a biological microscope, a stereoscopic microscope, and a phase-contrast microscope. However, it is not limited thereto.

According to another feature of the present disclosure, in the method for providing information about in vitro fertilized embryo screening according to an embodiment of the present disclosure, the convolutional neural network model pre-trained to determine the in vitro fertilized embryo with the converted original embryo image, the converted ICM extracted image, and the converted TE extracted image as inputs may be configured to further output the screening accuracy for the screening of the in vitro fertilized embryo.

According to another feature of the present disclosure, in the method for providing information about in vitro fertilized embryo screening according to an embodiment of the present disclosure, the convolutional neural network model may include one or more convolutional neural network models selected from VGG16, ResNet50, DenseNet121, EfficientNet, GoogLeNet, LeNet, AlexNet, U-Net, OverFeat, and ZFNet. However, it is not limited thereto.

In order to achieve the above-described objects, a device for providing information about in vitro fertilized embryo screening according to another embodiment of the present disclosure is provided. The device for providing information about in vitro fertilized embryo screening includes a communication unit configured to receive an original embryo image; a processor connected to the communication unit for communication; the processor is configured to extract, from the original embryo image, each of an inner cell mass (ICM) image and a trophectoderm (TE) image; convert each of the original embryo image, extracted ICM image, and extracted TE image into a binary image or grayscale image; and determine an in vitro fertilized embryo by using converted original embryo image, converted ICM extracted image, and converted TE extracted image as inputs, by using a convolutional neural network model pre-trained to determine an in vitro fertilized embryo by using the converted original embryo image, the converted ICM extracted image, and the converted TE extracted image as inputs.

According to a feature of the present disclosure, the processor may be configured to reconstruct the converted original embryo image, the converted ICM extracted image, and the converted TE extracted image into a single reconstructed image such that each of the images is distinguishable from one another, and determine the in vitro fertilized embryo with the reconstructed original embryo image, the reconstructed extracted ICM image, and the reconstructed extracted TE image as inputs.

According to another feature of the present disclosure, the communication unit further receives the mother's age value, and the processor may be configured to further include the mother's age value as an input value for the convolutional neural network model, and determine the in vitro fertilized embryo using the reconstructed original embryo image, the reconstructed extracted ICM image, the reconstructed extracted TE image, and the mother's age value as inputs.

According to another feature of the present disclosure, in the apparatus for providing information about in vitro fertilized embryo screening according to an embodiment of the present disclosure, the original embryo image may be an image captured by one or more microscopes selected from a biological microscope, a stereoscopic microscope, and a phase-contrast microscope. However, it is not limited thereto.

According to another feature of the present disclosure, in the apparatus for providing information about in vitro fertilized embryo screening according to an embodiment of the present disclosure, the convolutional neural network model pre-trained to determine the in vitro fertilized embryo with the converted original embryo image, the converted ICM extracted image, and the converted TE extracted image as inputs may provide further screening accuracy for the screening of the in vitro fertilized embryo.

According to another feature of the present disclosure, in the apparatus for providing information about in vitro fertilized embryo screening according to an embodiment of the present disclosure, the convolutional neural network model may be one or more convolutional neural network models selected from VGG16, ResNet50, DenseNet121, EfficientNet, GoogLeNet, LeNet, AlexNet, U-Net, OverFeat, and ZFNet. However, it is not limited thereto.

Hereinafter, the following embodiments are provided to further illustrate the present disclosure. However, these embodiments are merely illustrative of the present disclosure and should not be interpreted as limiting the scope of the present disclosure.

### Effects of the Invention

The present disclosure may provide information on the screening of an in vitro fertilized embryo by using a convolutional neural network model pre-trained to screen the in vitro fertilized embryo with the converted original embryo image, converted ICM extracted image, and converted TE extracted image as inputs, and furthermore provide information on the optimal screening of the in vitro fertilized embryo by using the convolutional neural network model additionally trained on images with adjusted Red, Blue, and Green values in RGB channel of each of the converted original embryo binary image, ICM extracted binary image, and TE extracted binary image so that each of the images is distinguishable from one another.

Through this, Researchers, including medical professionals and scientists who observe embryo images to determine whether in vitro fertilization has occurred may screen the embryo with the highest probability of pregnancy and implant it into the mother's uterus. Furthermore, the artificial intelligence model of the present disclosure may predict the pregnancy success rate.

Effects according to the present disclosure are not limited to the contents exemplified above, and more diverse effects are included in the present disclosure.

### BRIEF DESCRIPTION OF THE SEVERAL VIEWS OF THE DRAWINGS

FIG. 1a is an illustrative diagram of a system for providing information about in vitro fertilized embryo screening according to an embodiment of the present disclosure.

FIG. 1b is an illustrative diagram of the configuration of an apparatus for providing information about in vitro fertilized embryo screening according to an embodiment of the present disclosure.

FIG. 1c is an illustrative diagram of the configuration of a user device that receives and outputs information about in vitro fertilized embryo screening from an apparatus for providing information about in vitro fertilized embryo screening according to an embodiment of the present disclosure.

FIG. 2a is an illustrative diagram of the procedure for a method for providing information about in vitro fertilized embryo screening according to an embodiment of the disclosure.

FIG. 2b is an illustrative diagram of the procedure for the method for providing information about in vitro fertilized embryo screening according to an embodiment of the disclosure, illustrated using an embryo image implemented by a processor.

FIG. 3 illustrates a data collection and extraction process for establishing a convolutional neural network model according to another embodiment of the present disclosure.

FIG. 4 illustrates the types of input values for the convolutional neural network model according to another embodiment of the disclosure and the performance comparison results for in vitro fertilized embryo screening according to each type of convolutional neural network model.

FIG. 5 illustrates the statistical significance results of the mother's age as an input value for the convolutional neural network model according to another embodiment of the disclosure.

FIG. 6 is a diagram showing the main activation areas with high activation levels determined by the convolutional neural network model when the convolutional neural network model according to another embodiment of the present disclosure screens embryos that have successfully undergone in vitro fertilization as embryos that have successfully undergone in vitro fertilization.

FIG. 7 is a diagram showing the main activation areas with high activation levels determined by the convolutional neural network model when the convolutional neural network model according to another embodiment of the present disclosure screens embryos that failed in vitro fertilization as embryos that failed in vitro fertilization.

### DETAILED DESCRIPTION

The advantages and features of the present disclosure, and the methods for achieving them, will become clear with reference to the embodiments described in detail below together with the attached drawings. However, the present disclosure is not limited to the embodiments disclosed below, but may be implemented in various different forms, and these embodiments are provided only to make the disclosure of the present disclosure complete and to fully inform a person having ordinary knowledge in the technical field to which the present disclosure belongs of the scope of the disclosure. In relation to the description of the drawings, similar reference numerals may be used for similar components.

In the present disclosure, the expressions “have,” “can have,” “include,” or “can include” refer to the presence of a feature (for example, a numerical value, function, operation, or component such as a part) and do not exclude the presence of additional features.

In the present disclosure, the expressions “A or B,” “at least one of A or/and B,” or “one or more of A or/and B” may include all possible combinations of the items listed together. For example, “A or B,” “at least one of A and B,” or “at least one of A or B” may all refer to (1) including at least one A, (2) including at least one B, or (3) including both at least one A and at least one B.

The expressions “first,” “second,” “firstly,” or “secondly,” used in the present disclosure may describe various components, regardless of order and/or importance, and are used only to distinguish one component from another, but do not limit the components. For example, a first user device and a second user device may represent different user devices, regardless of order or importance. For example, without departing from the scope of the rights set forth in the present disclosure, a first component may be referred to as a second component, and similarly, a second component may be referred to as a first component.

When a component (for example, a first component) is referred to as being “(operatively or communicatively) coupled with/to” or “connected to” another component (for example, a second component), it should be understood that said component may be directly connected to said other component, or may be connected via another component (for example, a third component). Meanwhile, when it is said that a component (for example, a first component) is “directly connected” or “directly connected” to another component (for example, a second component), it can be understood that no other component (for example, a third component) exists between said component and said other component.

The expression “configured to” as used in the present disclosure can be used interchangeably with, for example, “suitable for,” “having the capacity to,” “designed to,” “adapted to,” “made to,” or “capable of,” depending on the situation. The term “configured (or set) to” does not necessarily mean that something is “specifically designed to” in terms of hardware. Instead, in some contexts, the expression “an apparatus configured to” can mean that the apparatus is “capable of” doing something in conjunction with other apparatus or components. For example, the phrase “a processor configured (or set) to perform A, B, and C” may mean a dedicated processor (for example, an embedded processor) for performing the operations, or a general-purpose processor (for example, a CPU or an application processor) that can perform the operations by executing one or more software programs stored in a memory device.

The terms used in the present disclosure are used only to describe specific embodiments and may not be intended to limit the scope of other embodiments. The singular expression may include the plural expression unless the context clearly indicates otherwise. Terms used herein, including technical or scientific terms, may have the same meaning as commonly understood by a person of ordinary skill in the art described in the present disclosure. Terms defined in general dictionaries among the terms used in the present disclosure may be interpreted as having the same or similar meaning as they have in the context of the relevant technology, and shall not be interpreted in an ideal or excessively formal sense unless explicitly defined in the present disclosure. In some cases, even if a term is defined in the present disclosure, it cannot be interpreted to exclude embodiments of the present disclosure.

Each feature of the various embodiments of the present disclosure can be partially or entirely combined or combined with each other, and as can be fully understood by those skilled in the art, various technical connections and operations are possible, and each embodiment can be implemented independently of each other or can be implemented together in a related relationship.

For clarity in the interpretation of this specification, the terms used in this specification are defined below.

The term “user” used in this document may refer to medical professionals and scientists who observe embryo images to determine whether in vitro fertilization has occurred, but is not limited thereto.

The term “embryo” as used herein may refer to an embryo having morphological characteristics consisting of at least 32 cells (blastocyst) after fertilization. Meanwhile, embryos may be obtained from mammals, including humans. Preferably, they may be human embryos.

Furthermore, the aforementioned orders of mammals include Monothematic, Metatheria, Didelphimorphia, Paucituberculata, Microbiotheria, Dasyuromorphia, Peramelemorphia, Notoryctemorphia, Diprotodontia, Insectivora, Macroscelidea, Scandentia, Dermoptera, Chiroptera, Primates, Xenarthra, Pholidota, Lagomorpha, Rodentia, Cetacea, Carnivora, Tubulidentata, Proboscidea, Hyracoidea, Sirenia, Perissodactyla, and Artiodactyla.

The term “in vitro fertilization (IVF)” used in this document refers to a procedure in which embryos formed by combining sperm and eggs in vitro are implanted into the mother's uterus to induce pregnancy, as a form of assisted reproductive technology (ART).

The term “inner cell mass (ICM)” used in this document is known as the embryoblast or morula, which is a cluster of cells inside the blastocyst of an early-stage embryo and may refer to the part that develops into a fetus after fertilization.

The term “trophectoderm (TE)” used in this document refers to the ectoderm of the trophectoderm, which surrounds the embryo. The trophectoderm mediates the attachment of the blastocyst to the uterine epithelium, supplies nutrients to the embryo, and may develop into many parts of the placenta.

The term “Convolutional neural network (CNN) model” used in this document, as a type of multilayer perceptron model that uses images as input, classifies the input images, and outputs the results, may be one or more models selected from VGG16, ResNet50, DenseNet121, EfficientNet, GoogLeNet, LeNet, AlexNet, U-Net, OverFeat, and ZFNet, and may be further configured into an ensemble model that trains weights between the probabilities of training models generated by multiple task execution nodes.

The term “gradient-weighted class activation map” used in this document may include major activation areas with high activation levels determined by the convolutional neural network model described in this document, and the major activation areas may be normalized to have values between 0 and 1 depending on their degree. Meanwhile, in this document, users may use the weighted class activation map to confirm the activated areas weighted by the convolutional neural network model according to an embodiment of the present disclosure when identifying in vitro fertilized embryos.

The term “channel” used in this document refers to the basic channel supported by graphics software such as Adobe Photoshop, which is an abbreviation of the first letters of Red, Green, and Blue, and may refer to a channel that stores the three-color mixture information of red, green, and blue in an embryo image.

The term “mother's age” used in this document refers to the age of the woman who provides the egg and receives the fertilized embryo. The mother's age value may be entered as a natural number in years.

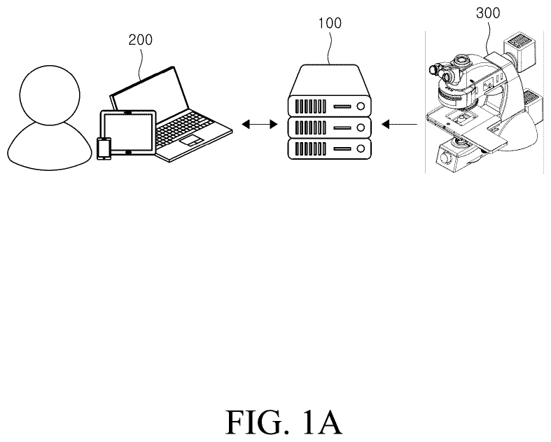

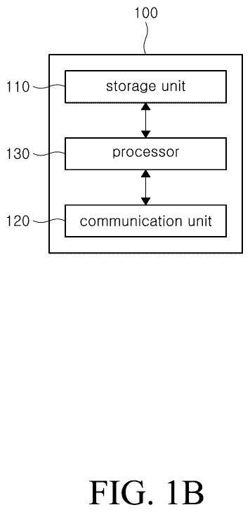

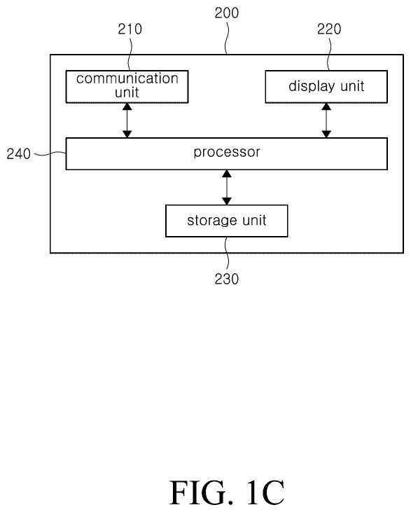

Below, with reference to FIGS. 1a to 1c, a system for providing information about in vitro fertilized embryo screening according to an embodiment of the present disclosure is described.

FIG. 1a is an illustrative diagram of a system for providing information about in vitro fertilized embryo screening according to an embodiment of the present disclosure.

FIG. 1b is an illustrative diagram of the configuration of an apparatus for providing information about in vitro fertilized embryo screening according to an embodiment of the present disclosure.

FIG. 1c is an illustrative diagram of the configuration of a user device that receives and outputs information on in vitro fertilized embryo screening from the apparatus for providing information about in vitro fertilized embryo screening according to an embodiment of the present disclosure.

With reference to FIG. 1a, a system (1000) for providing information about in vitro fertilized embryo screening may be configured with a user device (200), an apparatus (100) for providing information about in vitro fertilized embryo screening, and an apparatus (300) for capturing images of in vitro fertilized embryos.

Here, the user device (200), the apparatus (100) for providing information about in vitro fertilized embryo screening, and apparatus (300) for capturing images of in vitro fertilized embryos may be connected via a wired or wireless network.

First, the apparatus (100) for providing information about in vitro fertilized embryo screening may include a general-purpose computer, laptop, and/or data server that performs various operations to provide information about in vitro fertilized embryo screening using embryo image data transmitted from apparatus (300) for capturing images of in vitro fertilized embryos, and may preferably be an edge computing server. Furthermore, by including a display unit, information about in vitro fertilized embryo screening may be displayed to the user without any procedure for providing it to the user device (200).

The user device (200) is an electronic device for displaying information about in vitro fertilized embryo screening and may include at least one of a smartphone, tablet PC (Personal Computer), laptop, and/or PC.

Lastly, the apparatus (300) for capturing images of in vitro fertilized embryos may be one or more of a biological microscope, a stereoscopic microscope, and a phase-contrast microscope, but is not limited thereto. Furthermore, the format of embryo images captured by the apparatus (300) for capturing images of in vitro fertilized embryos may be one or more of BMP, JPG, PNG, and RGB color image file formats, but is not limited thereto, and preferably may be an RGB file format.

FIG. 1b is an illustrative diagram of the configuration of an apparatus for providing information about in vitro fertilized embryo screening according to an embodiment of the present disclosure.

With reference to FIG. 1b, the apparatus (100) for providing information about in vitro fertilized embryo screening includes a storage unit (110), a communication unit (130), and a processor (120).

First, the storage unit (110) may store various data related to in vitro fertilized embryo screening. For example, the storage unit (110) may store original embryo images captured by the apparatus (300) for capturing images of in vitro fertilized embryos, as well as images extracted, converted, or reconstructed by the processor (120).

Furthermore, the storage unit (110) may be configured to store data including images extracted by a convolutional neural network model, accuracy of embryo screening, and mother's age value.

In various embodiments, the storage unit (110) may include at least one type of storage medium selected from flash memory type, hard disk type, multimedia card micro type, card type memory (e.g., SD or XD memory, etc.), RAM, SRAM, ROM, EEPROM, PROM, magnetic memory, magnetic disk, and optical disk.

The communication unit (130) connects the apparatus (100) for providing information about in vitro fertilized embryo screening to an external device to enable communication. The communication unit (130) may connect to a user device (200) and further to the apparatus (300) for capturing images of in vitro fertilized embryos using wired or wireless communication to transmit and receive various data. Specifically, the communication unit (130) may receive embryo images from the apparatus (300) for capturing images of in vitro fertilized embryos. Furthermore, the communication unit (130) may transmit embryo images and information about in vitro fertilized embryo screening to the user device (200). Furthermore, by including a display unit, information about in vitro fertilized embryo screening may be displayed to the user without any procedure for providing it to the user device (200) (not shown).

The processor (120) is operably connected to the storage unit (110) and the communication unit (130) and may perform various commands for image analysis.

Specifically, the processor (120) may be configured to extract inner cell mass (ICM) images and trophectoderm (TE) images from the original embryo image based on the embryo image received through the communication unit (130); convert the original embryo image, the extracted ICM image, and the extracted TE image into binary images; reconstruct the converted original embryo image, ICM extracted image, and TE extracted image by adjusting the Red, Green, and Blue values of the RGB channel of each image so that each of the images are distinguishable from each other; reconstruct the original embryo image, ICM extracted image, and TE extracted image which are distinguished from each other by adjusting the Red, Green, and Blue values of the RGB channel into a single image; and screen an in vitro fertilized embryo by using the reconstructed images as inputs. In this case, the processor (120) may be based on a convolutional neural network model pre-trained to screen in vitro fertilized embryos from embryo images.

Meanwhile, the apparatus (100) for providing information about in vitro fertilized embryo screening is not limited to hardware-based designs. For example, the processor (120) of the apparatus (100) for providing information about in vitro fertilized embryo screening may be implemented as software. Accordingly, information about in vitro fertilized embryo screening may be displayed through the display unit of the apparatus (300) for capturing images of in vitro fertilized embryos connected to the aforementioned software.

FIG. 1c is an illustrative diagram of the configuration of the user device that receives and outputs information about in vitro fertilized embryo screening from the apparatus (100) for providing information about in vitro fertilized embryo screening according to an embodiment of the present disclosure.

With reference to FIG. 1c, the user device (200) includes a communication unit (210), a display unit (220), a storage unit (230), and a processor (240).

The communication unit (210) may be configured to enable the user device (200) to communicate with external devices. The communication unit (210) may be connected to the apparatus (100) for providing information about in vitro fertilized embryo screening using wired or wireless communication, and may transmit various data related to providing information about in vitro fertilized embryo screening. Specifically, the communication unit (210) may receive embryo-related visual information, including embryo images and videos, from the apparatus (100) for providing information about in vitro fertilized embryo, but is not limited thereto.

The display unit (220) may display various interface screens for displaying information about in vitro fertilized embryo screening. For example, the display unit (220) may display embryo images and, furthermore, display the accuracy of in vitro fertilized embryo screening. The accuracy may be indicated as a percentage and also be expressed as various images, including bar graphs or diagrams that indicate the accuracy.

In various embodiments, the display unit (220) may include a touchscreen and receive touch, gesture, proximity, drag, swipe, or hovering inputs using, for example, an electronic pen or a part of the user's body.

The storage unit (230) may store various data used to provide a user interface for displaying result data. In various embodiments, the storage unit (230) may include at least one type of storage medium among a flash memory type, a hard disk type, a multimedia card micro type, a card-type memory (e.g., SD or XD memory, etc.), RAM (Random Access Memory), SRAM (Static Random Access Memory), ROM (Read-Only Memory), EEPROM (Electrically Erasable Programmable Read-Only Memory), PROM (Programmable Read-Only Memory), magnetic memory, magnetic disk, or optical disk.

The processor (240) is operably connected to the communication unit (210), the display unit (220), and the storage unit (230), and may perform various commands to provide a user interface for displaying the result data.

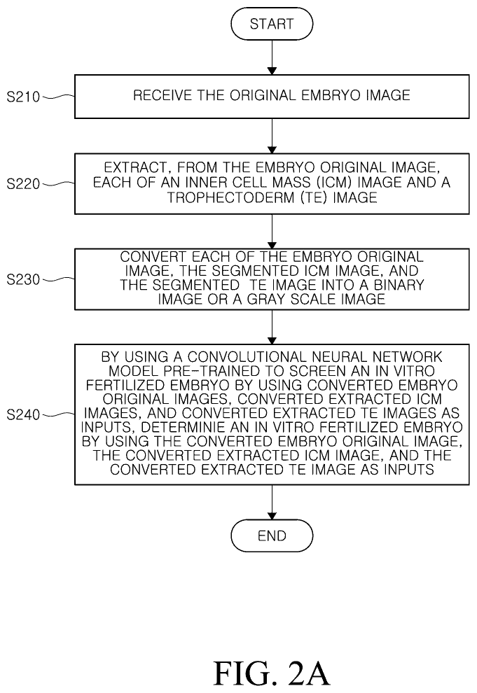

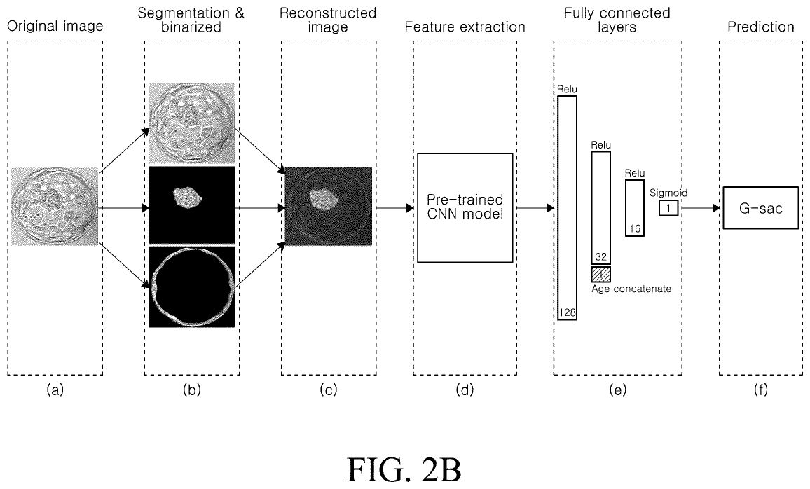

Below, with reference to FIGS. 2a to 2b, the method for providing information about in vitro fertilized embryo screening according to an embodiment of the present disclosure is described in detail.

FIG. 2a is an illustrative diagram of the procedure for a method for providing information about in vitro fertilized embryo screening according to an embodiment of the disclosure.

FIG. 2b illustrates the procedure for the method for providing information about in vitro fertilized embryo screening according to an embodiment of the disclosure using embryo images implemented by a processor.

For convenience of explanation, the reference numerals described in FIGS. 1a to 1c are used.

With reference to FIG. 2a, the procedure for providing information about in vitro fertilized embryo screening according to an embodiment of the present disclosure is as follows. The processor according to an embodiment of the present disclosure (1) receives an original embryo image (S210). The processor (2) extracts the inner cell mass (ICM) image and trophectoderm (TE) image from the received original embryo image (S220). The processor (3) converts the original embryo image, extracted ICM image, and extracted TE image into binary images or grayscale images (S230). The processor (4) finally, by using a convolutional neural network model pre-trained to screen an in vitro fertilized embryo by using the converted original embryo image, the converted ICM extracted image, and the converted TE extracted image as inputs, determines an in vitro fertilized embryo by using the converted original embryo image, the converted ICM extracted image, and the converted TE extracted image as inputs. (S240).

Hereinafter, for the description of FIG. 2a, reference will be made to FIG. 2b.

With reference to FIG. 2a and (a) of FIG. 2b, in the step (S210) where the processor (240) receives the original embryo image, the original embryo image may be an image captured using one or more microscopes selected from a biological microscope, a stereo microscope, and a phase-contrast microscope. The image format may be one or more of BMP, JPG, PNG, and RGB color image file formats, but is not limited thereto.

The method for extracting the ICM binary image and TE binary image from the original embryo image is not limited. The extraction may be performed using segmentation and may be performed using one or more convolutional neural network models selected from VGG16, ResNet50, DenseNet121, EfficientNet, GoogLeNet, LeNet, AlexNet, U-Net, OverFeat, and ZFNet.

Next, with reference to FIGS. 2a and (b) and (c) of FIG. 2b, when the processor reconstructs the converted original embryo image, ICM extracted image, and TE extracted image by adjusting the Red, Green, and Blue values of the RGB channel of each image so that each of the images are distinguishable from each other, the processor may reconstruct the extracted ICM binary image by adjusting the R, G, and B values of the RGB channels to 255, 0, and 0, respectively; and may reconstruct the extracted TE binary image by adjusting the R, G, B values of the RGB channels to 0, 255, and 0, respectively. Furthermore, the processor may be configured to screen an in vitro fertilized embryo using the reconstructed image as input. In this embodiment, reconstruction is described through adjustment of R, G, and B values, but is not limited thereto, the image may be reconstructed through adjustment of other specified color values.

Furthermore, with reference to FIGS. 2a and (b) and (c) of FIG. 2b, the processor may be configured to reconstruct the reconstructed original embryo image, the reconstructed ICM extracted image, and the reconstructed TE extracted image, which are distinguished from each other by adjusting the red, green, and blue values of the RGB channels, into one image; and to screen an in vitro fertilized embryo by using the reconstructed image as input.

Meanwhile, when training an ICM extracted binary image and a TE extracted binary image, the processor according to an embodiment of the present disclosure may be configured to perform training by creating a training image by adjusting the R value of the ICM extracted binary image and the G value of the TE extracted binary image, and by creating a training image by adjusting the G value of the same ICM extracted binary image and the R value of the same TE extracted binary image (i.e., reconstructing it to a different color than the original). In this case, substantially, the effect of adding weight to the extracted ICM image and extracted TE image rather than the original image may be produced.

With reference to (c) to (e) of FIG. 2b, the processor may include the mother's age value as an input value to the convolutional neural network model pre-trained the aforementioned reconstructed image.

Meanwhile, the convolutional neural network model according to an embodiment of the present disclosure is not significantly limited in terms of the specific model, and may be one or more convolutional neural network models selected from VGG16, ResNet50, DenseNet121, EfficientNet, GoogLeNet, LeNet, AlexNet, U-Net, OverFeat, and ZFNet. Preferably, the convolutional neural network model according to an embodiment of the present disclosure may be one or more convolutional neural network models selected from VGG16, ResNet50, and DenseNet121.

Furthermore, the convolutional neural network model of the present disclosure may provide greater accuracy for in vitro fertilized embryo screening. Furthermore, the accuracy may be expressed as a percentage and may also be represented by various images, such as bar graphs or diagrams that indicate the accuracy.

### Example: Collection of Original Embryo Image and Establishment of Prediction Model

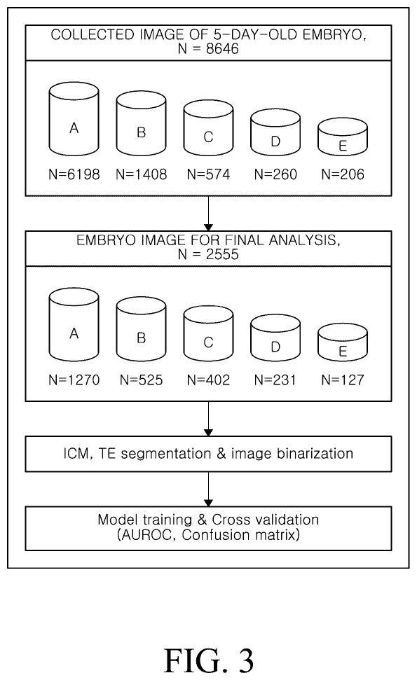

Hereinafter, with reference to FIG. 3, the data collection and extraction process for establishing a convolutional neural network model according to another embodiment of the present disclosure is described.

The inventors of the present disclosure collected a total of 8,646 images of 5-day-old embryos and related data, including the mothers' ages, from a total of seven in vitro fertilization clinics between June 2011 and May 2022 for the establishment of the convolutional neural network model of the present disclosure. The data collection and overall research design for this disclosure were conducted after IRB approval from all seven institutions participating in this study (IRB No. 2022-RESEARCH-01, GMH-2022-01, HIIRB 2022-01, RTR-2022-01, AJIRB-MED-MDB-21-716, 2204-003-113, and B-2208-7720194).

With reference to FIG. 3, among the seven institutions, four institutions (A, B, C, and D) had at least 200 embryo images each, but the remaining three institutions had insufficient numbers for statistical analysis, so they were combined into one institution (E) for analysis. A total of 2,555 embryo images, for which the success or failure of in vitro fertilization was confirmed, were used for the final analysis.

Furthermore, the inventors of the present disclosure extracted the ICM and TE regions from the collected 5-day-old embryo images, and the accuracy of the ICM and TE region extraction was finally confirmed through a secondary review by experienced clinical experts on the extracted image results. The inventors stored the coordinates of the ICM and TE regions obtained in this process in Json (JavaScript Object Notation) file format, then used OpenCV (Open-Source Computer Vision) to generate images of the ICM and TE regions, respectively, and then trained images of the ICM and TE regions to develop the final prediction model.

### Evaluation: Evaluation of Convolutional Neural Network Models According to Various Embodiments of the Present Disclosure

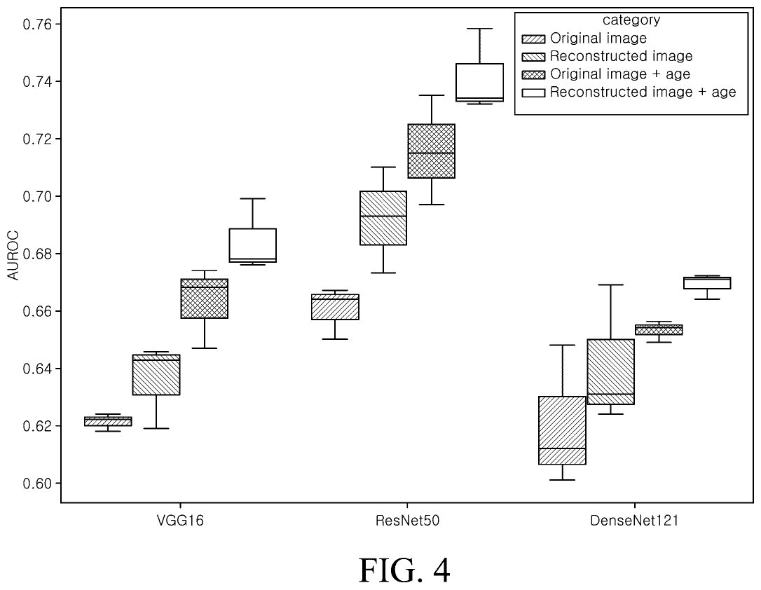

Hereinafter, with reference to FIGS. 4 to 7, the evaluation results of convolutional neural network models according to another embodiment of the present disclosure are described.

FIG. 4 shows the performance comparison results of in vitro fertilized embryo screening according to the types of input values and convolutional neural network models in another embodiment of the present disclosure.

First, with reference to FIG. 4, the convolutional neural network models used in this embodiment are VGG16, ResNet50, and DenseNet121 from the left side of the X-axis.

Furthermore, the Y-axis “AUROC” represents the area under the ROC curve when the performance comparison results of in vitro fertilized embryo screening using various convolutional neural network models are represented as a ROC curve. A high AUROC value may indicate that the model has excellent performance in distinguishing classes. Furthermore, the AUROC of the present disclosure may be expressed as the screening accuracy when screening the optimal in vitro fertilized embryo from the embryo images input to the convolutional neural network model of the present disclosure.

Meanwhile, in the category on the upper right side of FIG. 4, in the category at the top right of FIG. 4, “image” represents that according to another embodiment of the present disclosure, as input values for each model, the reconstructed embryo original image, the reconstructed ICM extracted image, the reconstructed TE extracted image, which are distinguished from each other by adjusting the Red, Green, and Blue values of the RGB channels, and the image in which the three images are reconstructed into one image were used as input values, “Reconstructed image+age” represents the input values for each model, including the maternal age value and the aforementioned reconstructed image, “Original image” represents Control Group 1, including the original embryo color image, the ICM extracted color image, and the TE extracted color image as input values for each model, “Original image+age” represents Control Group 2, including the mother's age value and the original embryo color image, the ICM extracted color image, and the TE-extract color image as input values for each model.

With reference to FIG. 4, when the input values for each model according to another embodiment of the present disclosure are Reconstructed image or Reconstructed image+age, all three convolutional neural network models (VGG16, ResNet50, and DenseNet121) show better performance than when original image or Original+age are used as input values.

In particular, the ResNet50 model using the Reconstructed image+age category as input values showed an AUROC average and standard deviation of 0.741 and 0.019, respectively, and an accuracy average and standard deviation of 0.682 and 0.015, respectively, showing superior classification performance compared to the DenseNet121 model and VGG16 model.

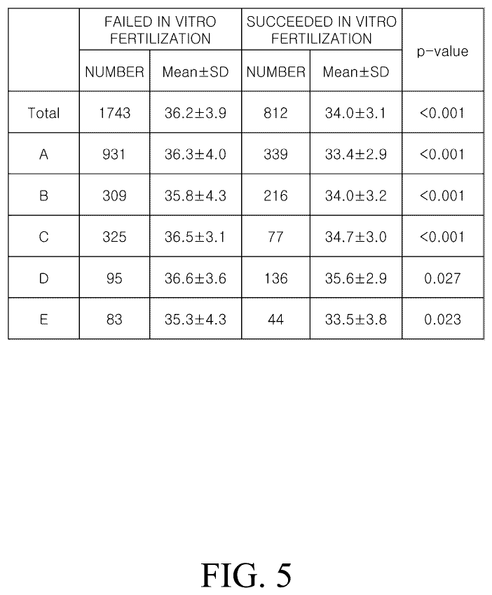

Meanwhile, the inventors of the present disclosure confirmed whether there was a significant difference in the age of the mother between the embryo images that were successfully fertilized in vitro and those that failed to be fertilized in vitro using a T-test (FIG. 5).

FIG. 5 shows the statistical significance results of the mother's age as input values for the convolutional neural network model according to another embodiment of the present disclosure.

With reference to FIG. 5, the average age of the mother group of embryos that failed in vitro fertilization was 36.2 years, which was significantly higher than the average age of 34.0 years of the mother group of embryos that succeeded in vitro fertilization.

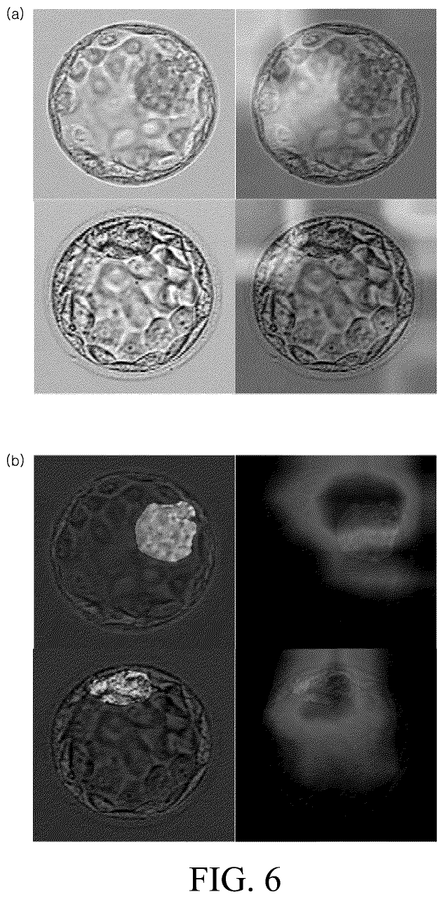

FIG. 6 is a diagram showing the main activation areas with high activation levels determined by the convolutional neural network model according to another embodiment of the present disclosure when screening embryos that succeeded in vitro fertilization as embryos that succeeded in vitro fertilization.

Specifically, in each of the four images in (a) of FIG. 6 and (b) of FIG. 6, the left two images represent the images input into the convolutional neural network model according to another embodiment of the present disclosure, i.e., the input values; the right two images among the four images in each of (a) of FIG. 6 and (b) of FIG. 6 represent the activated regions obtained by the convolution neural network model according to another embodiment of the present disclosure using a gradient-weighted class activation map to classify embryos successfully fertilized in vitro from the input images.

First, with reference to (a) of FIG. 6, the convolutional neural network model trained using the original color image of the in vitro fertilized embryo with the three RGB channels combined as input values to the convolutional neural network model (the left two images in (a) of FIG. 6) failed to identify the inner cell mass (ICM) region (see the right two images in (a) of FIG. 6). More specifically, as shown in the two images on the right side of (a) of FIG. 6, the convolutional neural network model trained using the original-color image of the embryo as input data marked regions other than the ICM region as active areas.

However, as shown in (b) of FIG. 6, the convolutional neural network model trained using the reconstructed image according to another embodiment of the present disclosure as the input to the convolutional neural network model accurately identified the inner cell mass (ICM) region from the reconstructed in vitro fertilized embryo image (the left two images in (b) of FIG. 6)(see the right two images in (a) of FIG. 6). More specifically, as shown in the right two images in (b) of FIG. 6, the convolutional neural network model trained using the reconstructed image according to another embodiment of the present disclosure as input indicates the ICM region as an activated area.

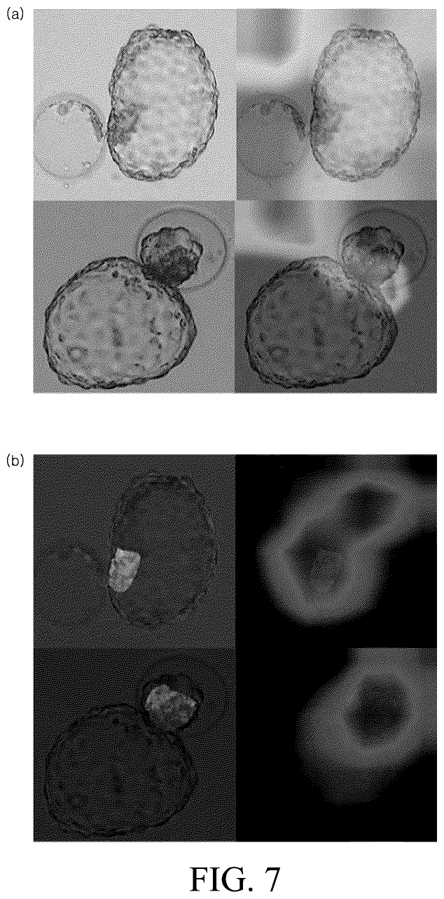

FIG. 7 is a diagram showing the main activation areas with high activation levels determined by the convolutional neural network model according to another embodiment of the present disclosure when screening embryos that failed in vitro fertilization.

Specifically, among the four images in each of (a) and (b) of FIG. 7, the left two images represent the images input to the convolutional neural network model according to another embodiment of the present disclosure, i.e., the input values; the right two images among the four images in each of (a) and (b) of FIG. 7 represent the activated regions obtained by the convolution neural network model according to another embodiment of the present disclosure using a gradient-weighted class activation map to classify the input images into embryos that failed in vitro fertilization.

First, with reference to (a) of FIG. 7, a convolutional neural network model trained with original color images of embryos that failed in vitro fertilization, with the three RGB channels combined as input values, failed to identify the inner cell mass (ICM) region from the original color images of embryos that underwent in vitro fertilization (the left two images in (a) of FIG. 7), with the three RGB channels combined as input values (see the right two images in (a) of FIG. 7). More specifically, as shown in the two images on the right side of (a) of FIG. 7, the convolutional neural network model trained using the original color images of embryos as input values marked regions other than the ICM region as active areas.

However, as shown in (b) of FIG. 7, the convolutional neural network model trained using the reconstructed image according to another embodiment of the present disclosure as the input value accurately identified the inner cell mass (ICM) region from the reconstructed embryo image that failed in vitro fertilization (the left two images in (b) of FIG. 7) (see the right two images in (a) of FIG. 7). More specifically, with reference to the right two images in (b) of FIG. 7, the convolutional neural network model trained using the reconstructed image as input according to another embodiment of the present disclosure indicates the ICM region as an active region even in embryos that failed in vitro fertilization.

That is, with reference to FIGS. 6 and 7, users may more accurately screen in vitro fertilized embryos from embryo images based on the convolutional neural network model of the present disclosure. Furthermore, through the convolutional neural network model of the present disclosure, it is possible to provide an environment where those suffering from infertility can receive optimal infertility treatment by implanting the most suitable in vitro fertilized embryos selected with greater accuracy into the mother's uterus.

Although the embodiments of the present disclosure have been described in more detail with reference to the attached drawings, the present disclosure is not necessarily limited to these embodiments, and various modifications may be made without departing from the technical idea of the present disclosure. Accordingly, the embodiments disclosed in the present disclosure are not intended to limit the technical idea of the present disclosure, but to explain it, and the scope of the technical idea of the present disclosure is not limited by these embodiments. Therefore, it should be understood that the embodiments described above are exemplary in all aspects and not restrictive. The protection scope of the present disclosure should be interpreted by the claims below, and all technical ideas within the equivalent scope should be interpreted as being included in the scope of the rights of the present disclosure.

### DESCRIPTION OF SYMBOLS

1000: System for providing information about in vitro fertilized embryo screening
100: Apparatus for providing information about in vitro fertilized embryo screening
110, 230: Storage unit
120, 240: Processor
130, 210: Communication unit
200: User device
220: Display unit
300: Apparatus for capturing images of in vitro fertilized embryos
[National Research and Development Project Supporting the Present disclosure]
[Project Unique Number] 1711197097
[Project Number] RS-2023-00254175
[Ministry Name] Ministry of Science and ICT, Ministry of Industry, Trade, and Energy, Ministry of Health and Welfare, Ministry of Food and Drug Safety
[Project Management (Specialized) Institution Name] Inter-Ministerial Comprehensive Medical Device Research and Development Project Team
[Project Title] Inter-Ministerial Comprehensive Medical Device Research and Development Project
[Research Project Name] Demonstration of Clinical Utility of Embryo Artificial Intelligence Solutions and Medical Device Certification
[Project Executing Institution Name] KAI HEALTH INC.
[Research Period] Apr. 1, 2023~Dec. 31, 2025

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for providing information about in vitro fertilized embryo screening using an embryo image implemented by a processor, comprising:
receiving an original embryo image;
extracting, from the original embryo image, an inner cell mass (ICM) image and a trophectoderm (TE) image;
converting each of the original embryo image, extracted ICM image, and extracted TE image into a binary image or a gray scale image; and
determining an in vitro fertilized embryo by using converted original embryo image, converted ICM extracted image, and converted TE extracted image as inputs, by using a convolutional neural network model pre-trained to screen an in vitro fertilized embryo by using the converted original embryo image, the converted ICM extracted image, and the converted TE extracted image as inputs.

2. The method of claim 1, further comprising reconstructing the converted original embryo image, the converted ICM extracted image, and the converted TE extracted image into single reconstructed images such that each of the images is distinguishable from one another, and
wherein the determining determines an in vitro fertilized embryo by using the reconstructed original embryo image, the reconstructed extracted ICM image, and the reconstructed extracted TE image as inputs.

3. The method of claim 1, further comprising receiving mother's age value;
wherein the convolutional neural network model further includes the mother's age value as an input value of the model, and
wherein the determining determines an in vitro fertilized embryo by using the reconstructed original embryo image, the reconstructed extracted ICM image, the reconstructed extracted TE image, and the mother's age value as inputs.

4. The method of claim 1, wherein the original embryo image is an image captured by one or more microscopes selected from a biological microscope, a stereoscopic microscope, and a phase-contrast microscope.

5. The method of claim 1, wherein the convolutional neural network model pre-trained to determine an in vitro fertilized embryo by using the converted original embryo image, the converted ICM extracted image, and the converted TE extracted image as inputs is configured to further output the screening accuracy for the screening of the in vitro fertilized embryo.

6. The method of claim 1, wherein the convolutional neural network model includes one or more convolutional neural network models selected from VGG16, ResNet50, DenseNet121, EfficientNet, GoogLeNet, LeNet, AlexNet, U-Net, OverFeat, and ZFNet.

7. An apparatus for providing information about in vitro fertilized embryo screening, the apparatus comprising:
a communication unit configured to receive an original embryo image;
a processor connected to the communication unit for communication;
the processor is configured to:
extract, from the original embryo image, each of an inner cell mass (ICM) image and a trophectoderm (TE) image;
convert each of the original embryo image, extracted ICM image, and extracted TE image into a binary image or grayscale image; and
determine an in vitro fertilized embryo by using converted original embryo image, converted ICM extracted image, and converted TE extracted image as inputs, by using a convolutional neural network model pre-trained to determine an in vitro fertilized embryo by using the converted original embryo image, the converted ICM extracted image, and the converted TE extracted image as inputs.

8. The apparatus of claim 7, wherein the processor is configured to:
reconstruct the converted original embryo image, the converted ICM extracted image, and the converted TE extracted image into single reconstructed images such that each of the images is distinguishable from one another, and determine an in vitro fertilized embryo by using the reconstructed original embryo image, the reconstructed extracted ICM image, and the reconstructed extracted TE image as inputs.

9. The apparatus of claim 7, the communication unit further receives mother's age value;
wherein the processor in configured to further include the mother's age value as an input value of the convolutional neural network model, and determine an in vitro fertilized embryo by using the reconstructed original embryo image, the reconstructed extracted ICM image, the reconstructed extracted TE image, and the mother's age value as inputs.

10. The apparatus of claim 7, wherein the original embryo image is an image captured by one or more microscopes selected from a biological microscope, a stereoscopic microscope, and a phase-contrast microscope.

11. The apparatus of claim 7, wherein the convolutional neural network model pre-trained to determine an in vitro fertilized embryo by using the converted original embryo image, the converted ICM extracted image, and the converted TE extracted image as inputs is configured to further output the screening accuracy for the screening of the in vitro fertilized embryo.

12. The apparatus of claim 7, wherein the convolutional neural network model is one or more convolutional neural network models selected from VGG16, ResNet50, DenseNet121, EfficientNet, GoogLeNet, LeNet, AlexNet, U-Net, OverFeat, and ZFNet.
